(19) United States
(12) Patent Application Publication
(10) Pub. No.: US 2005/0201566 A1
(43) Pub. Date: Sep. 15, 2005

(54) Electronically amplified stethoscope [E.A.S] (with optional recoding and data transfer capability)

(76) Inventor: Gregory Louis Hoover, Merritt Island, FL (US)

Correspondence Address: Gregory Louis Hoover, 211 McLeod St., Merritt Island, FL, 32953, US

(21) Appl. No.: 10/796,848

(22) Filed: Mar. 9, 2004

(51) Int. Cl. A61B 7/04; A61B 5/02

(52) U.S. Cl. 381/67; 600/528; 181/131

(57) ABSTRACT

The nature of the technical disclosures of my invention is that a battery powered electrical amplifier is combined with the typical stethoscope, such that by the use of a mini-microphone placed within the membrane of the stethoscope's drum, those sounds heard by the stethoscope are detected by the microphone, then fed to and through the amplifier (to increase the volume of the sounds heard) and reproduced for the user who listens, to the sounds by means of a headset, headphones, earphones, or ear-insert mini-speakers. An additional aspect of the advanced model of the device described above, includes a compact disc or digital recording system and “bass” and “treble” acoustic level adjustment control, to record those sounds picked up by the microphone, and to help clarify those sounds heard.

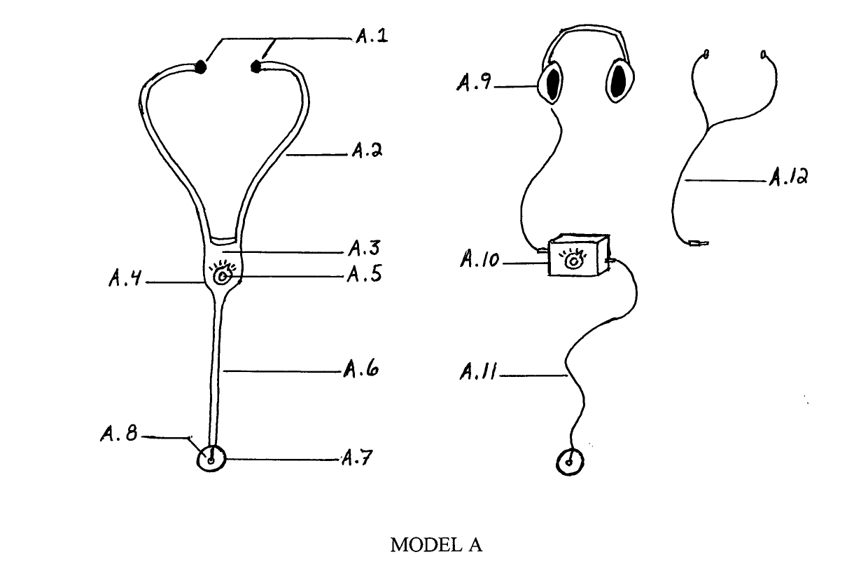

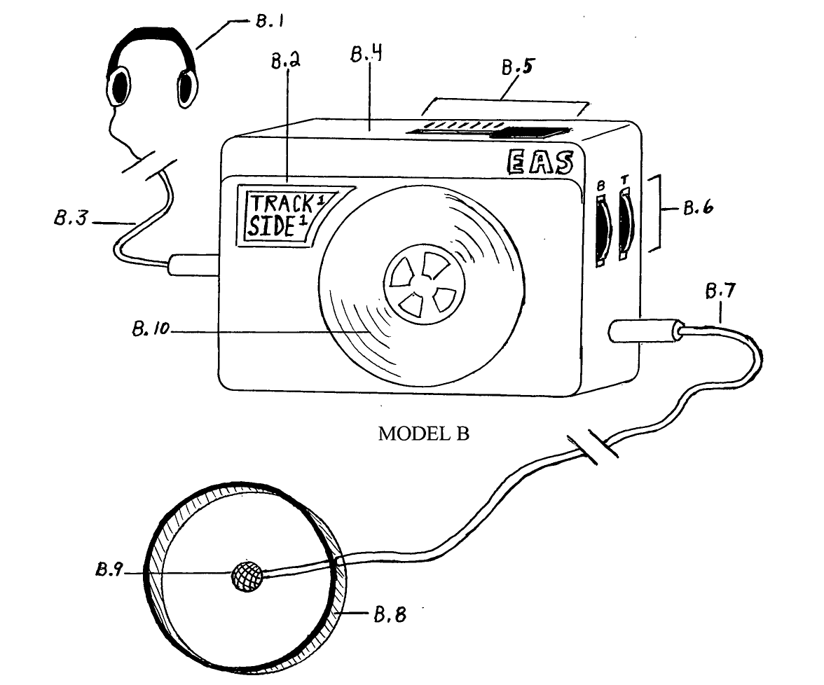

### STATEMENT REGARDING FEDERALLY SPONSORED RESEARCH OR DEVELOPMENT

This invention (the E.A.S.) was developed without the aid or assistance of Federal sponsorship.

### SPECIFICATION OF INVENTION

The named invention consists primarily of a microphoned drum, which feeds detected sound, via electrical wires, to an electrical amplifier, which by a battery power source, boosts said sounds in volume, which are then heard by the user, via electrically wired speakers in the form of a headset or ear-inserts. The “Advanced Model” of the invention additionally includes a digital or compact disc recording device which intercepts said detected sounds and records them for later replay or transference to any electronically powered data storage device (such as a computer).

### BACKGROUND OF THE INVENTION

The E.A.S. is the product of the inventor's intention to improve upon the pre-existing design and usefulness of the “stethoscope” as it is, and has been typically known. The claimed invention, in its' most basic proposed concept, is an improvement upon the traditional stethoscope, by boosting the volume of its detected sounds, and in its more advanced state, allowing for a recording (and storage) of such sounds.

The E.A.S. is intended to be used primarily in the field of medicine, but may be applied to other obvious uses.

### BRIEF SUMMARY OF THE INVENTION

The E.A.S. is an advancement upon the pre-existing non-electrical device commonly known as a “stethoscope”. The E.A.S. provides electrical amplification of those sounds typically detected by a stethoscope, using the addition of a battery powered microphone, amplifier, volume control and headset speaker or earphone/ear-piece system.

### BRIEF DESCRIPTION OF THE SEVERAL VIEWS OF THE DRAWINGS:

The inventor hereby further describes the E.A.S. by the use of several drawings which show both the basic model (labeled “Model A”) illustrated in two forms, and as well as the advanced model (labeled “Model B”) in an enlarged illustration.

Each drawing of the E.A.S. described above, includes alphanumerical notations which correspond to the following similarly listed items describing the areas, parts and/or functions referenced in said drawings.

Model A

As depicted by the illustrations labeled “Model A”.

A.1 Ear inserts with or housing earphones/ear speakers.

A.2 Electrically wired harness providing electrical signal or “feed” from the amplifier to the earphones.

A.3 Battery powered amplifier. To increase signal.

A.4 Battery compartment (behind panel on back or rear of amplifier).

A.5 Adjustable volume knob or dial. Used to increase or decrease the amplified signal or sound detected by the drummed microphone and fed to the earphones.

A.6 Electrically wired harness providing electrical signed or “feed” from the microphoned drum to the electric amplifier.

A.7 Drum with thin, sound sensitive plastic membrane, containing or housing miniature microphone.

A.8 Electronically wired miniature microphone used to detect sound vibrations within the drum (A.-7) and send signal to the electric amplifier.

A.9 Example of listening system shown as headset earphones (see also A.12).

A.10 Other example of electrical amplifier. This illustration of the unit shows the amplifier as a single component which allows for the use or replacement of separate headphone listening systems and harnessed drum (A.7), as well as permitting the unit to be worn by the user via a “clip-on” attachment.

A.11 Electronically wired harness providing electrical signal or feed from the microphoned drum to the amplifier, but represented as separate plug-in device.

A.12 Electronically wired harness providing electrical signal or feed from amplifier to earphone inserts, as opposed to headset, and as separate plug-in device.

Model B

As depicted by illustration labeled “Model B”.

B.1 Identical to that earlier described as depicted in A.1, A.2, A.9, and A.12.

B.2 L.E.D. “track” and “side” indicator denoting storage location of digitally recorded sounds or signals, detected, electronically by the device.

B.3 Plug-in portion of electrical wire leading from the E.A.S. to the headset or other listening device described earlier as depicted in A.1, A.2, and B.1.

B.4 The “advanced model” of the E.A.S. including a digital or compact disc recording system which provides for storage of the sounds detected by the E.A.S. for transference (via the compact disc, or direct digital relay) of such data to a computer data base, or by reference to the individual recordings retained upon the discs.

B.5 Sliding volume control or indicator. To be used to increase or decrease those sounds listened to via the E.A.S., as well as to adjust the volume of the detected sounds as recorded.

B.6 Bass and treble adjustment controls. To adjust the acoustic bass and treble levels for a refinement of the sounds heard and recorded.

B.7 Identical to that earlier described as depicted in A.11.

B.8 Identical to that earlier described as depicted in A.7.

B.9 Identical to that earlier described as depicted in A.8.

B.10 Compact disc data storage system used to record those sounds/signals detected by the E.A.S., for use as described earlier in B.4.

### DETAILED DESCRIPTION OF THE INVENTION

The Electronically Amplified Stethoscope (with optional compact disc or digital recording and data transfer capabilities) consists of a microphoned plastic membraned drum, which feeds detected sounds, via electrical wires, to an electric amplifier, which in turn, by a battery powered source, boosts said sounds in volume, so that said sounds are more easily and clearly heard by the user, who listens through an attached headset, earphones, or ear-insert mini-speakers. The advanced model of the basic concept provides the additional function of permitting the user to clarify the sounds with both bass and treble acoustic controls, as well as recording the detected sounds digitally or on compact disc.

The inventor requests the United States Patent and Trademark Office determine if this concept and device design is so novel as to be considered an original invention, or an “improvement” upon any previously patented device utilizing the traditional listening method provided by the common “stethoscope”.

While it is the inventor's desire and intention to be issued a Utility Patent on the described concept and device designs, should the USPTO determine same to be an “improvement” upon the common stethoscope or other previously patented improvements upon the stethoscope, the inventor desires that the USPTO receive and register the invention until such time that the USPTO makes that (“novel invention” or “improvement on a pre-existing invention”) determination, in a manner which assures and preserves the inventor's legal rights normally afforded under federal law.

The described invention is to be used primarily by and in the field of medicine, but also is directed to universal applications in any way the device(s) or concept may be used.

The concept of the device(s) is to include both the microphoned drum (illustrated and described as A.7, A.8, B.8, and B.9) and headphone or ear-insert speakers for listening purposes (illustrated and described as A.1, A.9, and B.1) to be attached to the main (E.A.S.) unit or device, via retractable (as by the use of a spring loaded spool) wires (described at A.2, A.12, and B.3), to be pulled out for use, then retracted back into the unit after use.

Several of the inventor's intended uses of the described invention, provide the following benefits:
1. The electronic amplification of those sounds typically intended to be detected and heard by physicians and others in the medical field (who presently use the common stethoscope) will permit those who are hearing impaired, to more clearly discern the sounds hoped to be heard.
2. The electrical amplification of the sounds heard by the user will allow for easier listening of those very slight and nearly inaudible sounds (such as the heartbeat of an unborn child) by a simple increase of the amplifier's volume control.
3. The electrical amplification of the sounds heard by the user, will permit a clearer hearing of such sounds in environments where background noise traditionally makes such listening more difficult (i.e., paramedics delivering medical aid at roadside—traffic conditions, and military medical personnel delivering aid under battlefield conditions).
4. The device's ability to offer the user an option of increasing either the bass or treble of the sounds heard will promote listening clarity.
5. The concept and described (as well as illustrated) invention in it's “advanced model” (depicted and described an “Model B”) provides the user the option of (by various methods) recording the sounds typically heard by the use of a common stethoscope. The invention in this form provides the user the additional benefit of storing such recorded sounds for later uses (i.e., doctors can keep files of patient heartbeats to record physical deterioration due to disease, or physical improvement due to medications, exercise or post-surgery). The stored recordings can later be analyzed by a physician's simply comparing more recent recordings to those recordings made during a patient's past examination. Such digital recordings may be fed into a computer which by an appropriate computer software program, may analyze such recordings to detect irregular sounds, so that the physician may be alerted to a potential ailment. Such digital recordings could be transmitted to or between physicians at differing locations [via internet].

1. What I claim as my invention is the electronic amplification of those sounds typically heard by the use of the common stethoscope, by means of a battery powered amplifier which boosts the volume of sounds detected by a miniature microphone located in the drum membrane of the stethoscope, that are then heard by the user via headphones, earphones, or ear-insert mini-speakers.

2. What I claim as my invention is an advanced model of the device described within the preceding paragraphed claim, being the addition of a compact disc or digital recording system to record those sounds detected by the use of the above described electronically amplified stethoscope.

3. What I claim as my invention is a “bass” and “treble” acoustic control mechanism to clarify the sounds detected (and or recorded) by the above described device.

4. What I claim as my invention is the described invention's providing digital recordings fed into a computer, which by an appropriate computer software program, may analyze such recordings to detect irregular sounds, so that a physician may be alerted to a potential ailment or the physical improvement of a patient.
